(19) United States
(12) Patent Application Publication
(10) Pub. No.: US 2012/0150062 A1
(43) Pub. Date: Jun. 14, 2012

(54) Non-Mercury Non-Electronic Clinical Thermometer with a Support Structure

(75) Inventor: Chu-Yih Yu, Taipei Hsien (TW)

(73) Assignee: MESURE TECHNOLOGY CO., LTD., San Chung City (TW)

(21) Appl. No.: 13/324,410

(22) Filed: Dec. 13, 2011

(30) Foreign Application Priority Data
Dec. 14, 2010 (TW) 099143799

(51) Int. Cl. A61B 5/01

(52) U.S. Cl. 600/549

(57) ABSTRACT

A thermometer includes a support structure and a heat conductive layer on an outer surface of the support structure. The heat conductive layer has a sensing portion and a temperature indication portion, and a structure with temperature indication markings is laid out along the temperature indication portion. A reversible temperature-sensitive color changing layer is formed on the temperature indication portion to overlap the structure of the temperature indication markings, in which the sensing portion is adapted for sensing a thermal contact surface of a user to conduct heat flow to the temperature-sensitive color changing layer, producing a sensed temperature and a color changing zone of the reversible temperature-sensitive color changing layer in response to the sensed temperature thereby exhibiting a corresponding temperature value.

### BACKGROUND OF THE INVENTION

1. Field of the Invention

The invention relates to the field of thermometers, and more particularly to the field of non-mercury non-electronic clinical thermometers with a support structure.

2. Description of the Related Art

For use in a health care field, a wide variety of materials and methods are available for detecting temperature and temperature changes. The most common example of a temperature indicating device is the mercury bulb thermometer. Bulb thermometers rely on the simple principle that the volume of a liquid expands upon heating and diminishes when cooled. Mercury bulb thermometers have become undesirable since mercury is highly toxic. In fact, many countries are actively banning or limiting the use of mercury and mercury thermometers.

Electronic thermometers generally offer a great number of advantages over conventional glass and mercury thermometers. Among the advantages of electronic thermometers are a digital temperature display to eliminate temperature reading errors; and with proper circuit design and calibration, higher accuracy and resolution is possible with accurate measurement and display of tenths of a degree Fahrenheit being easily attainable.

However, the above electronic thermometers are expensive since they require circuitry and/or processor chip. Furthermore, such electronic thermometers typically use a chemical battery as a power supply. In general, the electronic thermometers are idle for a long time since patients only use the electronic thermometers while they are sick or feel uncomfortable. Thus such electronic thermometers employing a chemical battery as a power supply are not environmentally friendly.

### SUMMARY OF THE INVENTION

An exemplary embodiment of the present invention overcomes the above-described problems by providing a thermometer includes a support structure and a heat conductive layer on an outer surface of the support structure. The heat conductive layer has a sensing portion and a temperature indication portion, and a structure with temperature indication markings is laid out along the temperature indication portion. A reversible temperature-sensitive color changing layer is formed on the temperature indication portion to overlap the structure of the temperature indication markings, in which the sensing portion is adapted for sensing a thermal contact surface of a user to conduct heat flow to the temperature-sensitive color changing layer, producing a sensed temperature and a color changing zone of the reversible temperature-sensitive color changing layer in response to the sensed temperature thereby exhibiting a corresponding temperature value.

### DESCRIPTION OF THE DRAWINGS

The present invention will be described by way of exemplary embodiments, but not limitations, illustrated in the accompanying drawings in which like references denote similar elements, and in which:

FIG. 1 is a schematic view of a heat conductive layer of a non-mercury non-electronic clinical thermometer according to an exemplary embodiment of the invention;

FIG. 2 is schematic view of a reversible temperature-sensitive color changing layer of the thermometer according to an exemplary embodiment of the invention;

FIG. 3 is a schematic view of a support structure of the thermometer according to an exemplary embodiment of the invention;

FIG. 4 is a schematic view of the thermometer according to an exemplary embodiment of the invention; and

FIG. 5 is a schematic view of a color changing zone of the thermometer according to an exemplary embodiment of the invention.

### DETAILED DESCRIPTION OF THE INVENTION

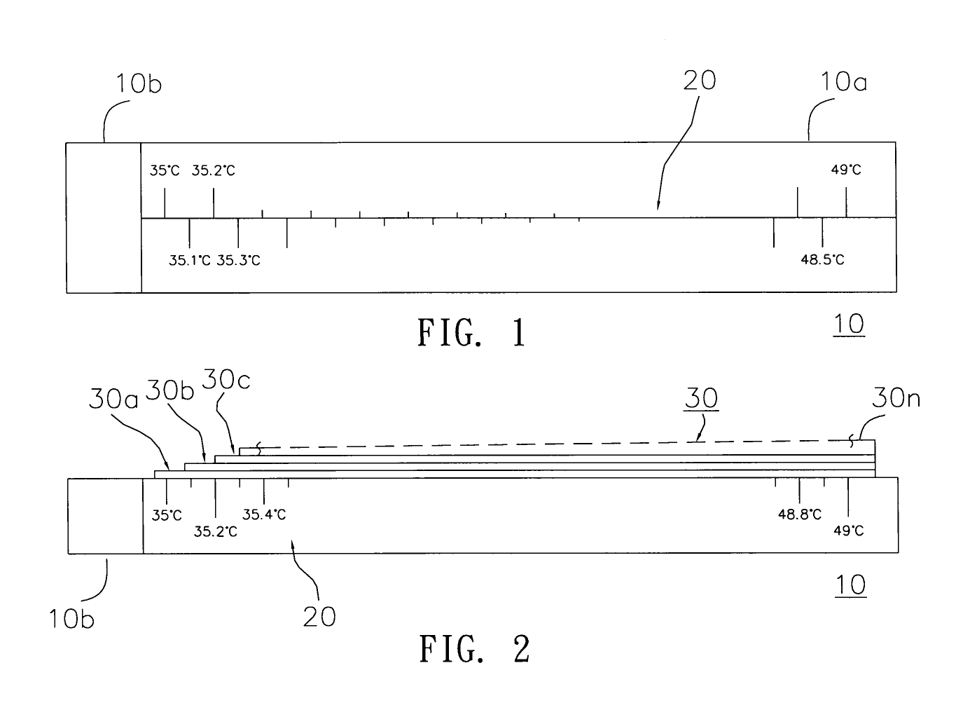

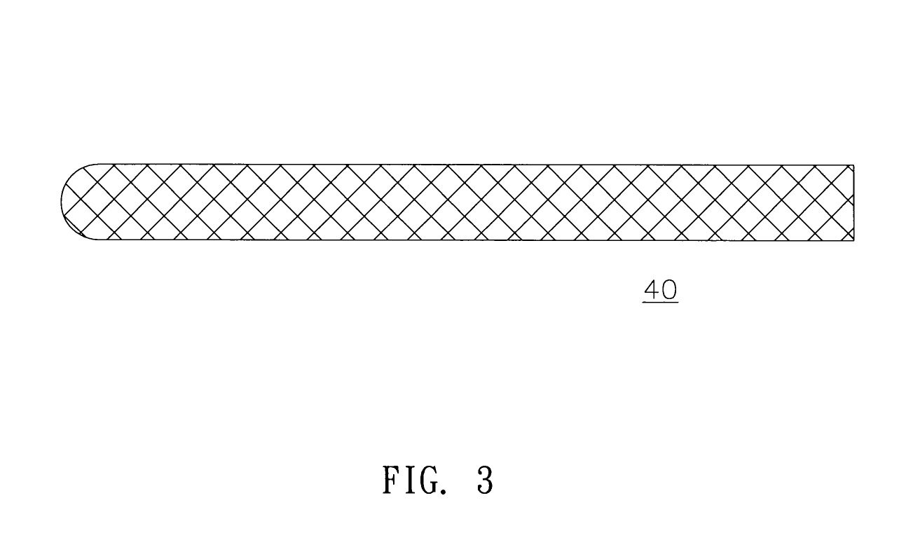

Referring to FIGS. 1 through 4, an embodiment of a thermometer 50 is illustrated. Thermometer 50 comprises a support structure 40, preferably, made of a metal material or a plastic material. In general, support structure 40 may be a hollow body such that a certain amount of heat capacitance could be reduced. Although optional, support structure 40 comprises a rounded front end and its surface is preferably made of a soft material.

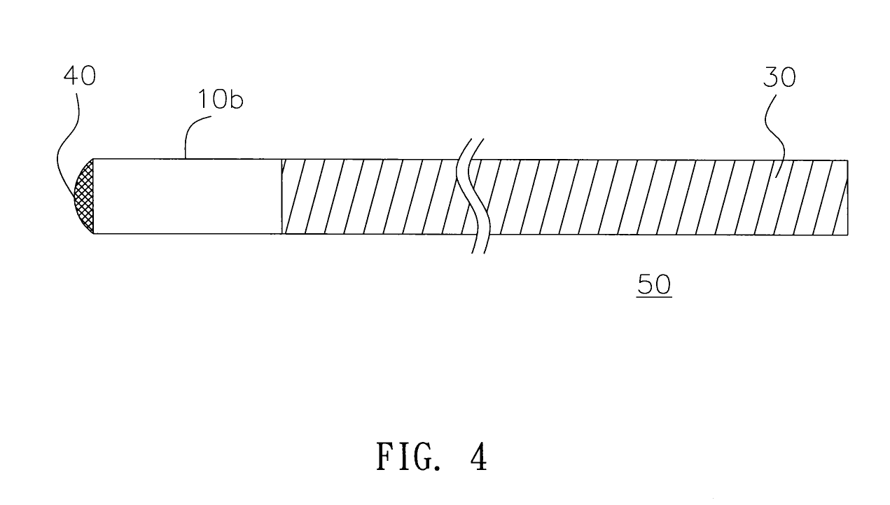

Next, as shown in FIGS. 1 and 4, a heat conductive layer is made up of a sensing portion 10b and a temperature indication portion 10a, generally formed on an outer surface of support structure 40. In one embodiment, sensing portion 10b is formed on or adjacent to the front end of support structure 40. The thermometer typically comprises a structure 20 with temperature indication markings laid out along temperature indication portion 10a, such as by means of coating or printing water-proof inks on or above a surface of temperature indication portion 10a of heat conductive layer 10.

In alternative embodiments, support structure 40 comprises a hollow tubular cylinder made of metal and its outer surface may directly serve as heat conductive layer 10. Support structure 40 can also comprises a hollow tubular cylinder made of plastic and its outer surface is further covered by a heat conducting film serving as heat conductive layer 10. Preferably, the heat conducting film is made of metal with good thermal conductivity.

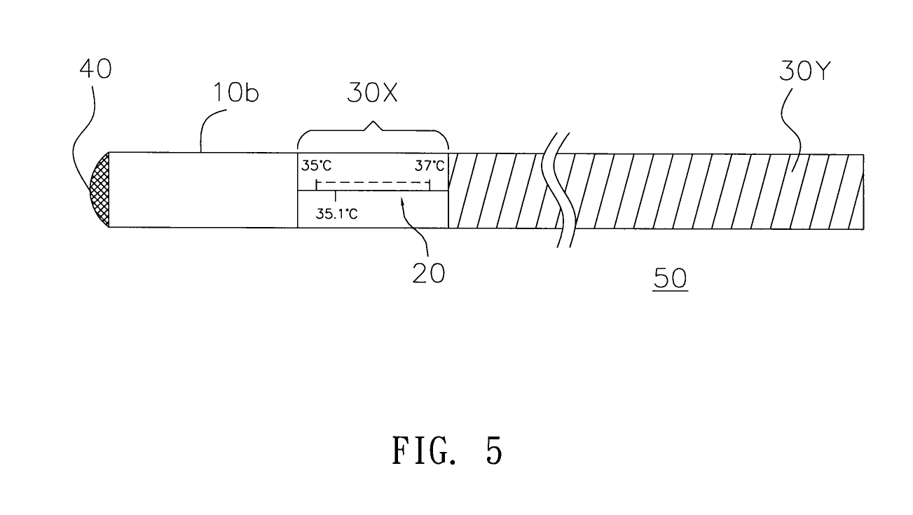

Turning to FIGS 2 and 4, a reversible temperature-sensitive color changing layer 30 is formed on temperature indication portion 10a to overlap structure 20 with the temperature indication markings. Specifically, temperature-sensitive color changing layer 30 has a first (original) color state in a prescribed temperature range. The first color state may be chosen to mask structure 20 with the temperature indication markings. In operation, sensing portion 10b is adapted for sensing a thermal contact surface of a user to conduct heat flow to temperature-sensitive color changing layer 30 on temperature indication portion 10a. As best shown in FIG. 5, a temperature is sensed and a color changing zone 30X with a second color state or a transparent state is produced in response to the sensed temperature thereby exhibiting a corresponding temperature value in structure 20 with the temperature indication markings. Furthermore, a remaining portion 30Y of temperature-sensitive color changing layer 30 keeps the original color state still unchanged since the sensed temperature is not enough to cause such state to appear.

Adverting to FIG. 2, temperature indication portion 10a comprises a proximate end adjacent sensing portion 10b and a distal end. Optionally, lower temperature values 30a in structure 20 with the temperature indication markings are marked on the proximate end adjacent sensing portion 10b, and higher temperature values 30n in structure 20 with the temperature indication markings are marked on the distal end. In one example, temperature-sensitive color changing layer 30 is divided into a plurality of blocks 30a-30n and each block corresponds a color-change temperature respectively. When in this configuration, sensing portion 10b is heated to conduct the heat flow to reversible temperature-sensitive color changing layer 30, and then the blocks with the color-change temperatures below the sensed temperature could change their original color state to the second color state or the transparent state such that color changing zone 30X is produced to exhibit the corresponding temperature value. Furthermore, color changing zone 30X may restore the first color state while the sensed temperature is gradually decreased below the color-changing temperatures of the sensed blocks.

As shown for instance in FIGS 4 and 5, assuming that the sensed temperature is 37° C. , color changing zone 30X comprising the blocks with color-changing temperatures below 37° C. changes its original color state to the transparent state. On the contrary, the remaining portion 30Y comprising the blocks with color-changing temperatures higher 37° C. maintains its original color state to mask the overlapped structure 20 with the temperature indication markings.

FIG. 2 illustrates a method of forming reversible temperature-sensitive color changing layer 30 over heat conductive layer 10. Specifically, reversible temperature-sensitive color changing layer 30 may be a multi-layer structure in which one block may be specified to have more layers for higher color-change temperature. For example, a first reversible temperature-sensitive color changing dye may be coated or printed on an entire surface of temperature indication portion 10a. Then, a second reversible temperature-sensitive color changing dye may be coated or printed on a portion of the surface of temperature indication portion 10a, such as covering the blocks 30b to 30n. In other words, the last reversible temperature-sensitive color changing dye may be coated or printed on the block 30n only. When in this configuration, the block 30a with a lowest color-change temperature has a thinnest thickness and the block 30n with a highest color-change temperature has a thickest thickness. Although optional, the multi-layer structure of reversible temperature-sensitive color changing layer 30 may be composed of several layers with different ingredients or doping densities.

In such thermometers, there is no need to use mercury and chemical battery which are easy to cause environmental pollution.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A thermometer comprising:
a support structure;
a heat conductive layer on an outer surface of the support structure, comprising a sensing portion and a temperature indication portion;
a structure with temperature indication markings, being laid out along the temperature indication portion; and
a reversible temperature-sensitive color changing layer, formed on the temperature indication portion to overlap the structure of the temperature indication markings, wherein the sensing portion is adapted for sensing a thermal contact surface of a user to conduct heat flow to the reversible temperature-sensitive color changing layer, producing a sensed temperature and a color changing zone of the reversible temperature-sensitive color changing layer in response to the sensed temperature thereby exhibiting a corresponding temperature value.

2. The thermometer as recited in claim 1 wherein the support structure comprises a body made of metal.

3. The thermometer as recited in claim 1 wherein the support structure comprises a hollow tubular cylinder made of metal and the heat conductive layer is the outer surface of the support structure.

4. The thermometer as recited in claim 1 wherein the support structure comprises a hollow tubular cylinder made of plastic and the heat conductive layer is a heat conducting film covering the hollow tubular cylinder.

5. The thermometer as recited in claim 1 wherein the support structure comprises a rounded front end adjacent to the sensing portion of the heat conductive layer.

6. The thermometer as recited in claim 5, wherein the rounded front end comprises a surface made of a soft material.

7. The thermometer as recited in claim 1, wherein the temperature-sensitive color changing layer has a first color state in a prescribed temperature range.

8. The thermometer as recited in claim 7, wherein the first color state is employed to mask the structure of the temperature indication markings.

9. The thermometer as recited in claim 7, wherein the color changing zone comprising a plurality of blocks with color-changing temperatures has a second color state or a transparent state exhibiting the temperature value, while the sensed temperature is higher than the color-changing temperatures of the blocks in the color changing zone.

10. The thermometer as recited in claim 9, wherein the first color state is restored while the sensed temperature is decreased below the color-changing temperatures.
